(19) United States
(12) Patent Application Publication
(10) Pub. No.: US 2026/0026688 A1
(43) Pub. Date: Jan. 29, 2026

(54) Wireless Interrogated Pressure Sensors, Readout Scheme, and Method of Manufacture

(71) Applicant: University of South Florida, Tampa, FL (US)

(72) Inventors: Ramesh Ayyala, Tampa, FL (US); Liguan Li, Tampa, FL (US); Jing Wang, Tampa, FL (US)

(21) Appl. No.: 19/279,910

(22) Filed: Jul. 24, 2025

Related U.S. Application Data
(60) Provisional application No. 63/675,001, filed on Jul. 24, 2024.

(51) Int. Cl. A61B 3/16; A61B 5/00

(52) U.S. Cl. CPC A61B 3/16; A61B 5/0004; A61B 5/0031; A61B 5/686; A61B 2562/0247; A61B 2562/164

(57) ABSTRACT

Disclosed herein are implantable sensors, including intraocular (IOP) pressure sensors suitable for continuous pressure monitoring and point-of-care applications, and methods of manufacture and use. In some implementations, the incorporation of repeater coil(s) as intermediate relays and addition of soft-magnetic ferrite materials to enhance magnetic resonance is strategically designed to result in improved signal strength while relaxing the needed accuracy in terms of position and angle alignment tolerance.

### CROSS-REFERENCE TO RELATED APPLICATIONS

This application claims priority to and the benefit of U.S. Provisional Application No. 63/675,001, titled “DESIGN OF WIRELESS INTERROGATED MEMS CAPACITTIVE INTRAOCULAR PRESSURE SENSORS”, filed on Jul. 24, 2024, the content of which is incorporated by reference herein in its entirety.

### BACKGROUND

Existing intraocular pressure (IOP) sensors and telemetry circuits typically have a short working distance and require a stringent external readout circuit to sensor alignment. For example, the quality factor of the planar spiral inductors of the sensor is often low and limits the resolution of the IOP sensors. Further, the IOP sensor external readout that is used to measure the frequency and phase response is often bulky and expensive which prevents adoption of the technology for point-of-care applications.

As such, there is a need for sensors and systems that are suitable for point-of-care applications by patients. These needs and others are at least partially satisfied by the present disclosure.

### SUMMARY

Embodiments of the present disclosure present the design and modeling of a Micro-Electro-Mechanical System (MEMS) based capacitive IOP sensor, which holds great potential as an implantable and battery-less sensor mounted inside, for example, an FDA-approved minimally invasive glaucoma surgery (MIGS) device for monitoring of intraocular pressure. A novel readout circuit scheme that is well suited for the point-of-care of outpatient usage cases is disclosed herein, which include the unique repeater circuit integrated into an oscillator for point-of-care readout from an implanted IOP sensor, and innovative usage of soft-magnetic ferrites to strengthen the magnetic coupling, and additional implant related bio-compatibility and surgical related techniques.

Design, simulation and optimization of both the external readout circuit and the proposed implantable inductor-and-capacitor (LC) tank circuit have been conducted. A variety of designs and layouts for both pressure sensitive MEMS membrane capacitor and integrated on-chip inductor has been explored. A micro-fabrication process flow is also included that enables batch fabrication and hermetic packaging of the proposed IOP sensor to operate as minimally invasive and implantable device within the inner eye. The proposed sensor can be adapted for real-time and continuous pressure monitoring in other parts of a subject's body including the brain and bladder, for example.

An example method includes surgical insertion of a MEMS intraocular pressure (IOP) sensor microchip implanted in the anterior chamber of the eye or other regions of a subject's eye. A study was conducted to assess an external readout coil and vector network (impedance) analyzer which can be replaced by a handheld close-loop oscillator-based mixed signal processor that can display the eye pressure in mmHg unit (after rigorous calibration procedure or novel in-situ self-test/calibration technique) and a readout circuit for accurate IOP reading to enable point-of-care testing by patients. The incorporation of repeater coil(s) as intermediate relays and addition of soft-magnetic ferrite materials to enhance magnetic resonance (also often referred as strongly coupled magnetic resonance) is strategically designed to result in improved signal strength while relaxing the needed accuracy in terms of position and angle alignment tolerance and increasing the distance between the implantable pressure sensor and external readout. The unique criteria to fulfill such magnetic resonance conditions would also enable us to carry out sensor calibration by following a novel calibration procedure. Also, a handheld close-loop oscillator-based mixed signal processor can be utilized to display the eye pressure in mmHg unit that facilitates patient self-testing scenario.

In one embodiment, a wireless interrogated Micro-electromechanical Systems (MEMS) capacitive intraocular pressure sensor is provided. The proposed pressure sensor can consist of an inductor-and-capacitor (LC) tank circuit constituted of a fixed on-chip spiral inductor and a pressure-sensitive variable capacitor with two parallel electrodes and four side supporting tethers, although the physical implementation of membrane-based MEMS variable capacitor can take different device structure and geometrical design (e.g., square, rectangular, or circular membrane shape, a parallel combination of an array of MEMS variable capacitors, and different supporting anchor or tether design, the employment of dimples or other anti-stiction features in membrane, different hermetic packaging configurations, etc.). An exemplary membrane dimension is 500 μm×500 μm×2 μm along with a MEMS capacitive transducer air gap of 2.5 μm. Depending on the needed pressure range and limit of detection, the exact membrane shape and dimensions would be tailored designed. Intuitively, larger membrane size (lateral dimensions) and thinner membrane thickness would result in improved pressure sensitivity and limit of detection (lowest detectable pressure change) and reduced operation pressure range (highest pressure a sensor can handle). Once the pressure is applied on the surface of the top membrane, the induced deformation will result in a proportional variation in the capacitance. This capacitance change will lead to a change in the amplitude and phase of frequency of the LC-tank circuit, which can be used to monitor the intraocular pressure (IOP) in a real-time and continuous fashion with a target detection range of 0 mmHg to 50 mmHg or higher along with a limit of detection (also known as resolution) better than 0.1 mmHg. These aforementioned performance parameters are chosen for the target IOP pressure monitoring scenario, which can be adjusted through design of MEMS membrane sensor in terms of membrane shape (e.g., circular, square, rectangular, etc.), lateral dimensions and thickness of the membrane, structural material of the membrane, thickness of the air gap (typically defined by the thickness of the sacrificial layer). Once IOP induced LC circuit change is noted, the information can be sent to an external readout system through mutual coupling between the inductors present inside the sensor and the external readout circuit.

In some implementations, a wireless intraocular pressure sensing system is provided. The system can include: a pressure sensor including a sealed or encapsulated parallel-plate capacitor with a deformable membrane, the pressure sensor being configured to be implanted in a subject's eye; an external reader (e.g., reader coil) outside the subject's eye configured to wirelessly interrogate the pressure sensor and detect changes in resonant frequency and phase corresponding with an induced deformation of the membrane resulting in capacitance changes corresponding to intraocular pressure variations; at least one repeater coil or circuit positioned (e.g., at optimal distances) between the implanted pressure sensor and the external reader configured to improve signal strength between the pressure sensor and the external reader, wherein the external reader includes a readout circuit (e.g., readout inductor coil positioned outside the subject's eye) and a signal processing circuit (e.g., closed loop oscillator) configured to: convert the detected changes in resonant frequency and phase to pressure readings, and output or transmit an indication of the pressure readings.

In some implementations, the pressure sensor includes an LC tank circuit including a spiral inductor and a variable capacitor connected in series or parallel circuit configuration, or an inductor electrically coupled to the capacitor to form a resonant LC circuit.

In some implementations, the external reader is configured to wirelessly interrogate the LC tank circuit or resonant LC circuit via inductive coupling.

In some implementations, the capacitance changes lead to a change in frequency and phase of the LC tank circuit or resonant LC circuit.

In some implementations, the signal processing circuit is configured to output or transmit the indication of the pressure readings to a computing device or graphical user interface.

In some implementations, the computing device is configured to transmit a control indication to cause an implanted glaucoma drainage device to drain fluid from the subject's eye.

In some implementations, the signal processing and readout circuit includes a closed-loop oscillator (e.g., in the place of a vector network analyzer or impedance analyzer) to form a point-of-care portable test module.

In some implementations, the system further includes: a soft-magnetic ferrite material (with high magnetic permeability and low magnetic loss tangent in either a thin sheet or other geometry) positioned between the implanted pressure sensor and the external reader to enhance magnetic coupling.

In some implementations, the pressure sensor is a Micro-Electro-Mechanical Systems (MEMS) capacitive pressure sensor.

In some implementations, the MEMS capacitive pressure sensor defines an array of pressure sensors to form a parallel array of varying-gap capacitors (unit cells).

In some implementations, the pressure sensor includes a biocompatible passivation layer (e.g., parylene).

In some implementations, the pressure sensor can be microfabricated with a wide range of structural materials (e.g., nickel, copper, gold, silicon, polymers, etc.) compatible with MEMS microfabrication processes/suitable for micromachining.

In some implementations, the pressure sensor is embedded in or embodied as a glaucoma drainage device or intraocular lens.

In some implementations, an operational distance between the pressure sensor and the external reader is up to 1 foot (e.g., between 0 cm and 1 foot).

In some implementations, the system detection range is between 0 mmHg and 50 mmHg, and the system resolution is at least 0.1 mmHg. These aforementioned performance parameters are chosen for the target IOP pressure monitoring scenario, which can be adjusted through design of MEMS membrane sensor in terms of membrane shape (e.g., circular, square, rectangular, etc.), lateral dimensions and thickness of the membrane, structural material of the membrane, thickness of the air gap (typically defined by the thickness of the sacrificial layer).

In some implementations, the pressure sensor is configured for implantation via at least one of: iris suturing, vitreous cavity anchoring, or a lens bag placement.

In some implementations, a method of fabricating the pressure sensor of any of the above embodiments is provided. The method can include: patterning a bottom electrode; depositing a plurality of layers (e.g., sacrificial layer, seed layer) on the bottom electrode; annealing a planar spiral inductor; integrating the planar spiral inductor surrounding or between the bottom electrode and a top electrode; and hermetically sealing the pressure sensor, wherein an air gap is present between the bottom electrode and the top electrode.

In some implementations, the pressure sensor is hermetically sealed using parylene C or other bio-compatible thin film deposition.

In some implementations, annealing the planar spiral inductor includes using rapid thermal processor (RTP) annealing or localized annealing through Joule heating via applied current.

In some implementations, a method of using the pressure sensor according to any of the embodiments described above is provided. The method can include: implanting the pressure sensor into a subject's eye; wirelessly interrogating the pressure sensor using the external reader; detecting changes in resonant frequency and phase corresponding to intraocular pressure variations; determining pressure readings from the detected changes in resonant frequency and phase, and outputting or transmitting an indication of the pressure readings. Optionally, the method includes carrying out sensor calibration by following a novel calibration procedure.

In some implementations, the pressure sensor is implanted via at least one of: iris suturing, vitreous cavity anchoring, or a lens bag placement.

In some implementations, a pressure sensing system is provided. The system can include: a pressure sensor including a sealed or encapsulated parallel-plate capacitor with a deformable membrane, the pressure sensor being configured to be implanted in a subject's body; an external reader configured to wirelessly interrogate the pressure sensor and detect changes in resonant frequency and phase corresponding with an induced deformation of the membrane resulting in capacitance changes corresponding to intraocular pressure variations; and at least one repeater coil or circuit positioned between the pressure sensor and the external reader configured to improve signal strength between the pressure sensor and the external reader, wherein the external reader includes a signal processing circuit configured to: convert the detected changes in resonant frequency and phase to pressure readings, and output or transmit an indication of the pressure readings

In some implementations, the pressure sensor is configured for bladder, intracranial, or cerebrospinal fluid (CSF) pressure monitoring.

In some implementations, the signal processing circuit is configured to output or transmit the indication of the pressure readings to a computing device or graphical user interface.

In some implementations, the system further includes: a soft-magnetic ferrite material (with high magnetic permeability and low magnetic loss tangent in either a thin sheet or other geometry) positioned between the implanted pressure sensor and the external reader to enhance magnetic coupling.

In some implementations, the pressure sensor includes an LC tank circuit including a spiral inductor and a variable capacitor connected in series or parallel circuit configuration, or an inductor electrically coupled to the capacitor to form a resonant LC circuit.

In some implementations, the external reader is configured to wirelessly interrogate the LC tank circuit or resonant LC circuit via inductive coupling.

In some implementations, the capacitance changes lead to a change in frequency and phase of the LC tank circuit or resonant LC circuit.

In some implementations, the signal processing circuit is configured to output or transmit the indication of the pressure readings to a computing device or graphical user interface.

In some implementations, the signal processing circuit and readout circuit includes a closed-loop oscillator.

In some implementations, the pressure sensor is a Micro-Electro-Mechanical Systems (MEMS) capacitive pressure sensor.

In some implementations, the MEMS capacitive pressure sensor defines an array of pressure sensors.

In some implementations, the pressure sensor includes a biocompatible passivation layer (e.g., parylene).

In some implementations, the pressure sensor can be microfabricated with a wide range of structural materials (e.g., nickel, copper, gold, silicon, polymers, etc.) compatible with MEMS microfabrication processes.

In some implementations, an operational distance between the pressure sensor and the external reader is up to 1 foot, although the extension of the external reader distance beyond 10 cm is anticipated to offer diminishing benefits for targeted point-of-care or outpatient applications.

In some implementations, the system detection range is between 0 mmHg and 50 mmHg, and wherein the system resolution is at least 0.1 mmHg. These aforementioned performance parameters are chosen for the target IOP pressure monitoring scenario, which can be adjusted through design of MEMS membrane sensor in terms of membrane shape (e.g., circular, square, rectangular, etc.), lateral dimensions and thickness of the membrane, structural material of the membrane, thickness of the air gap (typically defined by the thickness of the sacrificial layer).

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

The components in the drawings are not necessarily to scale relative to each other. Like reference, numerals designate corresponding parts throughout the several views.

FIG. 1A depicts the equivalent circuit for the proposed intraocular pressure sensor design (direct inductive coupling readout without a repeater coil), in accordance with certain embodiments of the present disclosure.

FIG. 1B is a circuit diagram showing an example resonant repeater circuit that includes a varactor capacitor and planar inductor, in accordance with certain embodiments of the present disclosure.

FIG. 1C is a circuit diagram showing an example reader circuit, in accordance with certain embodiments of the present disclosure.

FIG. 1D depicts a fully integrated readout circuit with a closed loop oscillator as signal processor and direct readout unit, in accordance with certain embodiments of the present disclosure.

FIG. 1E is a graph demonstrating inductance vs. the number of turns of readout coil inductors.

FIG. 1F are graphs showing measurement and simulation results of the readout coil inductor.

FIG. 1G is a graph demonstrating that the quality factor of the planar spiral inductor can be improved by annealing.

FIG. 2A and FIG. 2B show exemplary fabrication processes, for an exemplary pressure sensor, in accordance with certain embodiments of the present disclosure.

FIG. 2C is a flowchart diagram depicting an example method, in accordance with various embodiments described herein.

FIG. 3A is a zoom-in image of a fabricated IOP capacitive membrane sensor, in accordance with various embodiments described herein.

FIG. 3B depicts an initial sensor test and calibration scheme.

FIG. 3C shows a measured frequency response of the proposed IOP sensor during the initial testing before the calibration is applied.

FIG. 3D shows an extracted resonance frequency variation in response to liquid pressure change during the initial testing before the calibration is applied.

FIG. 3E is a schematic diagram of a LC passive wireless sensor with a repeater strategically applied and posited between the IOP sensor and external readout coil for relaxing the sensitivity of readout coil to IOP sensor alignment while fortifying output signal.

FIG. 3F are graphs showing tested magnetic permeability and loss tangent of NiCoZn soft magnetic ferrite sheet that can be used along with repeaters to enhance sensor readout via strongly coupled magnetic resonance.

FIG. 4 is an example computing device.

### DETAILED DESCRIPTION

It is appreciated that certain features of the disclosure, which are, for clarity, described in the context of separate embodiments, can also be provided in combination with a single embodiment. Conversely, various features of the disclosure, which are, for brevity, described in the context of a single embodiment, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

Throughout the description and claims of this specification, the word “comprise” and other forms of the word, such as “comprising” and “comprises,” means including but not limited to, and are not intended to exclude, for example, other additives, segments, integers, or steps. Furthermore, it is to be understood that the terms comprise, comprising, and comprises as they relate to various embodiments, elements, and features of the disclosure also include the more limited embodiments of “consisting essentially of” and “consisting of.”

As used herein, the singular forms “a,” “an,” and “the” include plural referents unless the context clearly dictates otherwise. Thus, for example, reference to a “polymer” includes embodiments having two or more such polymers unless the context clearly indicates otherwise.

Ranges can be expressed herein as from “about” one particular value and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It should be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

For the terms “for example” and “such as,” and grammatical equivalences thereof, the phrase “and without limitation” is understood to follow unless explicitly stated otherwise.

Glaucoma is a progressive disease that damages the optic nerve of the eye, and it is usually associated with elevated intraocular pressure that causes irreversible vision loss. Glaucoma is the second leading cause of visual impairment and blindness in the world [1]. One in every 40 adults, who are more than 40 years old, has glaucoma [2]. Current glaucoma treatments are designed to reduce intraocular pressure (IOP). There are many options for reducing IOP, including medications, laser treatments, and surgery. Current surgical treatments for glaucoma are aimed at reducing intraocular pressure by reducing the aqueous humor inflow or increasing the aqueous humor outflow. For instance, trabeculectomy is a standard surgical procedure for glaucoma patients, but it is usually reserved for moderate to severe disease. Over the past decade, new FDA-approved medical devices called minimally invasive glaucoma surgical (MIGS) devices have been deployed. These devices are designed to treat milder glaucoma by enhancing physiological humor outflow while minimizing tissue damage. As clinicians are satisfied with the use of drainage implants in refractory glaucoma, people are exploring the use of these devices for initial surgery trials. In randomized clinical trials, glaucoma drainage implants are proven to be superior to trabeculectomy and are gradually being accepted as the option for primary glaucoma surgery [3]. Increased clinical experience and improved surgical techniques have led to the popularity of glaucoma drainage implants. Since 2003, the number of glaucoma MIGS incisions performed annually in the United States has remained relatively stable, ranging from 55,000 to 61,000 [4]. On the contrary, the use of trabeculectomy decreased by 77% from 1994 to 2012. Glaucoma drainage implants largely compensated for the difference, with a 410% increase in use over the same period. The ratio of trabeculectomy to glaucoma drainage implants dropped from 27:1 in 1994 to 3:2 in 2012 [4]. According to National Institutes of Health (NIH), there are about 120,000 Americans suffered from glaucoma with vision loss, which constitutes 9 to 12 percent of blind people in the US. In 2010, there are 60.5 million glaucoma patients, and the expected amount will reach 79.6 million by 2020 around the world [5]. Even though surgery is deemed as the most popular treatment, it is also considered as a temporary solution with a risk of scar tissue buildup that increases the intraocular pressure. Thus, there is a great need for constant monitoring of the IOP since the long-term increase of the IOP can damage the optic nerve.

In this work, a wirelessly interrogated MEMS-based capacitive pressure sensor is designed and implemented to monitor the intraocular pressure. The proposed pressure sensor consists of a varying-gap capacitor that monitors the changes in capacitance as a function of pressure, which is integrated with a fixed inductor to create a mutual coupling between the implantable IOP sensor and the external readout unit. The pressure-induced capacitance variation results in a resonant frequency drift of the LC tank circuit, which can be used to monitor the intraocular pressure in a real-time and continuous fashion with a detection range of 0 mmHg to 50 mmHg along with a resolution better than 0.1 mmHg, where mmHg is the most widely used IOP unit. According to glaucoma research foundation, the normal eye pressure range is 12-22 mmHg [6]. Eye pressure greater than 22 mmHg or lower than 12 mmHg is considered abnormal, since the abnormal inner eye pressure is a significant risk factor. More importantly, lingering high eye pressure greater than 22 mmHg may cause glaucoma. Thus, the proposed MEMS pressure sensor should detect a wider range of eye pressure, ranging from 0 mmHg to 50 mmHg. By using a finite clement method (FEM) simulation tool (CoventorWare, ANSYS, COMSOL), a MEMS capacitive sensor model is built with strategically designed membrane sizes and material properties. Based on the initial design optimization, electroplated nickel can be employed as the structural material for the suspended membrane, while other alternative materials can be employed for microfabrication of varying-gap MEMS capacitor. Furthermore, the equipment circuit model of a co-fabricated on-chip spiral inductor is incorporated to form the battery-less LC tank circuit, which will be coupled with a set of external inductor coils as its readout circuit. This overall system consists of an LC tank circuit for the implantable sensor and an external readout coil, which are simulated in Advanced Design System (ADS). Meanwhile, a bench-top prototype system based on the same LC resonant coupling concept is implemented to evaluate the optimal readout scheme between the amplitude and phase of the LC resonant responses. In one embodiment, the target distance between the inductor in readout unit and inductor in pressure sensor is 5 cm.

In 2010, Chen et al. used a variable inductor surrounded by a capacitor to achieve a higher-pressure sensitivity of 695 ppm/mmHg, where the variable inductor had dual layer of coil. Parylene C was used as a biocompatible material to create flexible coil substrate, which can be folded subsequently for minimally invasive implantation [7]. In 2016, Zeng et al. used graphene to form the LC tank circuit [8]. The deformation of the membrane in the capacitor is being controlled by internal and external pressure. When the external pressure is increased, the top electrode on the membrane is pushed away causing a decrease in the measured capacitance. However, the detection distance is too short. In our proposed novel solution, two new strategies: a) the incorporation of repeater coil(s), and b) addition of soft-magnetic ferrite materials, can be strategically designed to result in improved signal strength while relaxing the needed accuracy in terms of position and angle alignment tolerance and increasing the distance between the implantable pressure sensor and external readout. This facilitates patient self-testing scenario.

In 2001, another design of a wireless batch scaled absolute capacitive pressure sensor was reported [9]. In this work, Akar et al. proposed an IOP sensor, which used a variable capacitor integrated with a gold-electroplated planar coil inductor to achieve a maximum value of Q of the sensor coil is 8 at the operational frequency. Thereafter, Chen et al. proposed a microfabricated implantable parylene-based wireless passive intraocular pressure sensors in 2008 [10]. In this work, the quality factor value of the IOP sensor is only 3. It is obviously that the quality factor of the IOP sensor is quite low, which needs to be improved. In our work, We use annealing (rapid thermal processor (RTP) annealing or localized annealing through Joule heating via applied current to improve the quality factor. In 2013, a study of a minimally invasive implantable wireless pressure sensor for continuous IOP monitoring was reported [11]. The phase response of the coil is monitored using an impedance analyzer by measuring the shift of resonant frequency. In 2017, Joohee et al. reported a wearable smart sensor systems integrated on soft contact lenses for wireless ocular diagnostics [12]. The vector network analyzer is used to measure the frequency response. Obviously, the impedance analyzer and vector network analyzer that they used to measure the frequency response are too bulky. We strive to develop the integrated readout circuit for accurate reading of the intraocular pressure induced response of MEMS capacitive membrane sensor that can eliminate the need of a bulky and costly vector network or impedance analyzer for the point-of-care measurement at home that has never been explored and demonstrated in prior works to our best knowledge. Without critical point-of-care, portable apparatus, it is impossible for real patients at home to use this technology as they need the help of engineers to operate specialized test tools (e.g., vector network analyzer/impedance analyzer). Fully integrated readout circuit with a closed-loop oscillator as signal processor and direct readout unit will be implemented to replace the bulky instruments (VNA or impedance analyzer) to support the target point-of-care applications

### Example System and Intraocular Pressure Sensor

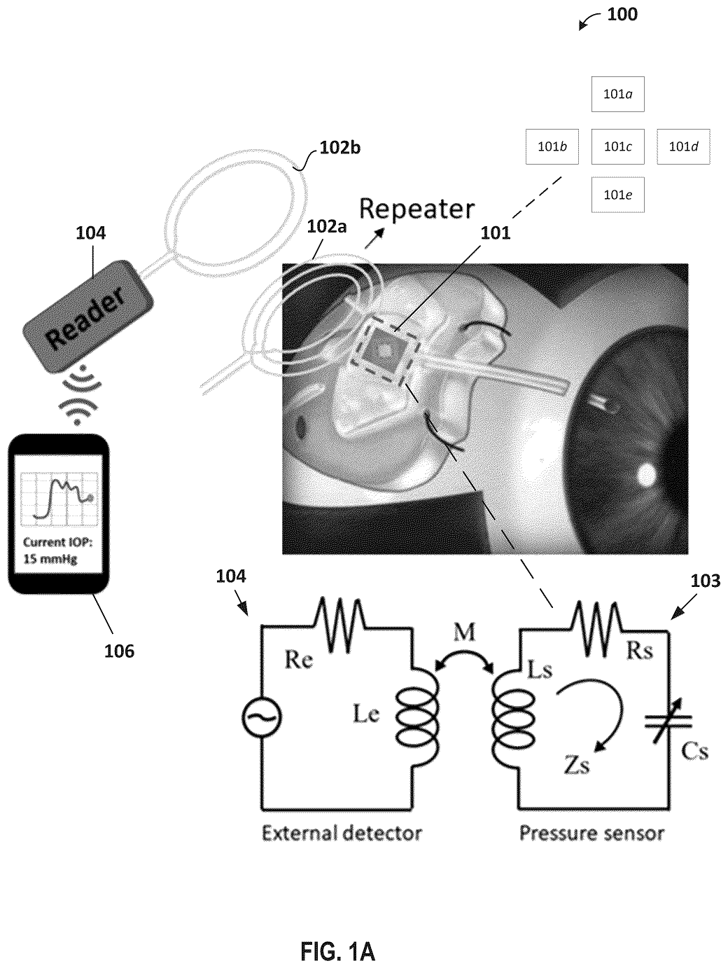

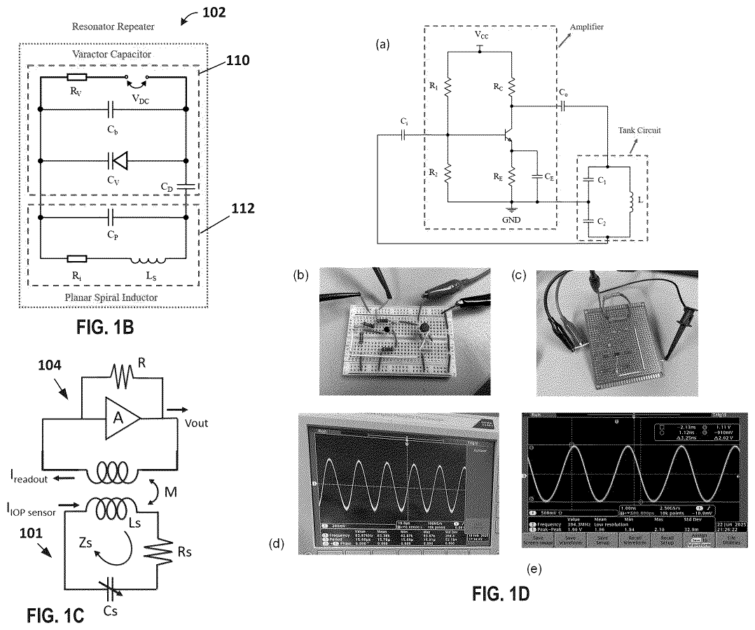

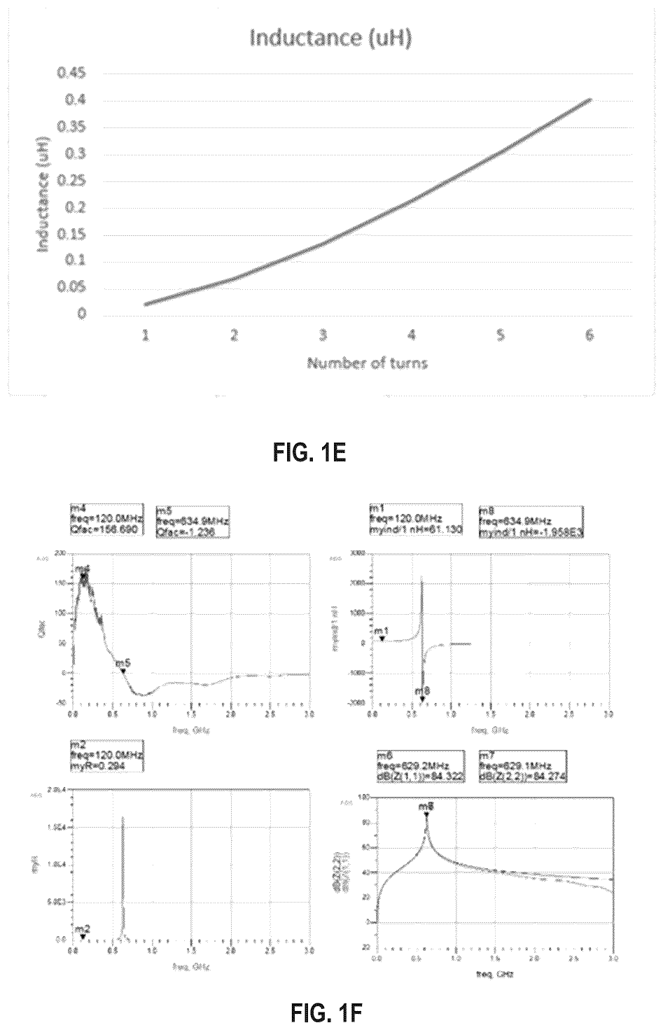

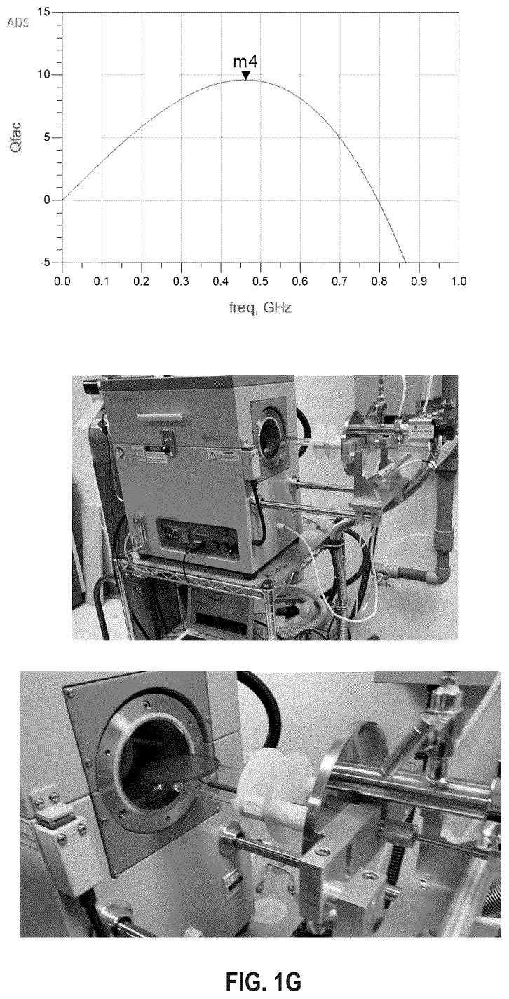

Referring now to FIG. 1A, an example system 100 is shown to include an implantable IOP sensor 101 that includes incorporate repeater coil(s) 102a, external readout coil(s) 102b and soft-magnetic ferrite materials to improve signal strength, relax positioning and alignment constraints, and increase the distance between the implantable pressure IOP sensor 101, external readout coil(s) 102b, and an external readout/reader 104 to facilitate point-of-care testing, for example, via a computing device 106. In some implementations, the quality factor of the planar spiral inductor can be improved by annealing (rapid thermal processor (RTP) annealing or localized annealing through Joule heating via applied current). A fully integrated readout circuit 104 (including the external readout coil(s) 102b) with a closed-loop oscillator as signal processor and direct readout unit is employed to replace conventional bulky instruments (e.g., vector network analyzer (VNA) or impedance analyzer). As illustrated, the sensor 101 can be or comprise an array of sensors 101a, 101b, 101c, 101d, 101e. A respective sensor 101 may be positioned in each of the subject's eyes.

As illustrated in FIG. 1A, the example IOP sensor 101 consists of two components: a fixed spiral inductor (Ls) and a variable capacitor (Cs), which form as an LC tank circuit. The capacitor is composed of two parallel plates, a fixed bottom electrode, and a movable pressure-sensitive membrane (top electrode). For the proposed design, the top electrode of the membrane capacitor deforms under the influence of the intraocular pressure. Meanwhile, the induced deformation changes the air gap between the two parallel membranes of the varying-gap MEMS capacitor, thus resulting in a capacitance change. The design with a smaller air gap is anticipated to exhibit a higher capacitance value that typically leads to a greater change in capacitance across the targeted intraocular pressure range of 0 mmHg to 50 mmHg. FIG. 1A depicts the equivalent circuit 103 for the proposed intraocular pressure sensor design. Moreover, Near Field Communication (NFC) or other wireless communication protocols can be used to transfer data from the implantable pressure sensor to an external readout circuit as shown in FIG. 1A [12].

FIG. 1A shows the equivalent circuit model of the inductive coupling link used in the telemetric readout. The sensor is modeled with a fixed inductor Ls, a series resistor Rs, and a variable capacitor Cs. The resonance frequency of the sensor is given by [12].

$${f}_{0} = \frac{1}{2 π \sqrt{LsCs}} \qquad ( 1 )$$

The resonance frequency changes in response to pressure, and it can be detected by inductive telemetry with an external coil inductor. Through inductive coupling, the external coil energizes the sensor circuit. M represents mutual inductance.

$$M = k \sqrt{LeLs} \qquad ( 2 )$$

where k is the coupling coefficient, and Le is the inductance of the external coil.

By monitoring the overall impedance change of the external coil due to the reflected impedance, it is possible to detect the sensor resonance frequency. The approximate magnitude of the impedance phase dip is given by:

$${Δφ}_{DIP} ≅ {tan}^{- 1} ( \frac{{ω}_{0} {M}^{2}}{LeRs} ) \qquad ( 3 )$$

where ω₀ is the angular resonance frequency, Rs is a series resistance of the sensor, M is the mutual inductance, and Le is the inductance of the external readout coil.

The impedance phase dip is maximized when the series resistance of the electroplated coil Rs is minimized, and its inductance Ls is maximized for larger M. However, it is important to note that, when Ls is increased by increasing the number of turns in the coil, then the parasitic capacitance of the coil also increases, decreasing the self-resonance frequency of the planar coil. The self-resonance frequency should be much higher than the operating frequency for proper device operation. It is also important to decrease the series resistance of the electroplated coil for a more accurate detection of the resonance frequency of the sensor. Low series resistance (thus high Q) inductors can be implemented by depositing thick metal films with low specific resistivity.

FIG. 1B is a circuit diagram showing an example resonant repeater circuit 102 that includes a varactor capacitor 110 and planar spiral inductor 112.

FIG. 1C is a simplified circuit diagram showing an example reader circuit 104 and IOP sensor circuit 101.

FIG. 1D depicts a fully integrated readout circuit with a closed loop oscillator as signal processor and direct readout unit. Panel (a) is a schematic diagram of a Colpitts oscillator; panel (b) shows the Colpitts oscillator implemented on breadboard; panel (c) shows the Colpitts oscillator implemented on a PCB board; panel (d) shows the Colpitts oscillator's output waveforms measured by an oscilloscope; and panel (c) shows the Colpitts oscillator's output waveforms measured by an oscilloscope.

### MEMS Membrane Capacitor Design

The design and analysis of MEMS membrane capacitor was done using CoventorWare (ANSYS or COMSOL can be used for this multi-physics simulation as well). Copper is chosen as the material of capacitors due to its high conductivity. A wide variety of membrane sizes, shapes, thicknesses, and air gap spacings were taken into account while designing the MEMS capacitor. In addition to FEM analysis by using CoventorWare, closed-form analytical calculations were performed.

The formulas for the maximum displacement and capacitance are shown below [13]:

$${W}_{max} = C \frac{1 - {v}^{2}}{{Eh}^{3}} {pb}^{4} \qquad ( 4 )$$

where C=0.032/(1+α4), α=b/a=1 (a =b), and p is the applied pressure. E is Young's modulus, ν is Poisson's ratio, h is the thickness of the top membrane, and b is the length of the top membrane.

$$Δ C = 0.0746 {C}_{0} p \frac{1 - {v}^{2}}{E} \frac{{a}^{4}}{{h}^{3} d} \qquad ( 5 )$$

$$C = Δ C + {C}_{0} \qquad ( 6 )$$

where C0=εA/d, d is the height of gap, and a is the half length of the top membrane.

As the external pressure is applied on the top membrane, the capacitive membrane is deformed. The deformation of capacitance will result in a change in the resonance frequency.

### Integrated Spiral Inductor Design

The integrated spiral inductor is designed and simulated using a 2.5 dimensional (2.5D) electromagnetic simulator (i.e., momentum in Advanced Design Systems (ADS)). The design procedure started by varying the number of turns and the shape of the inductor including length, width, spacing, and thickness. Nickel is chosen as the material of inductor because of the biocompatibility [14]. Also, nickel is non-magnetic without interference with sensitive electronics. The square shape of the inductor is chosen because it gives higher inductance. Spiral inductors are widely used in RF IC models as they give high inductance and are also compatible with the IC layout tool. Increasing the number of turns leads to a high inductance due to the increase of length.

As discussed above, the longer the total length is, the higher the effective inductance value will be. To get a longer inductor coil while efficiently using the chip area, the coil can be placed surrounding the membrane capacitor. As increasing number of turns within the specific chip area increases the parasitic capacitance in between turns, which will degrade the performance. There are many factors that need to be considered when designing the inductor, such as the number of turns, spacing, width, length, and so on. The quality factor is a very important parameter in the design of spiral inductor, which is one of the key elements to obtain a high-performance RF circuit.

The inductance and quality factor (Q) are given by [15]:

$$Ls = \frac{{μ}_{0} {n}^{2} {D}_{avg} {c}_{1}}{2} ( ln ( \frac{{c}_{2}}{ρ} ) + {c}_{3} ρ + {c}_{4} {ρ}^{2} ) \qquad ( 7 )$$

$$Q = \frac{2 π {f}_{0} {L}_{s}}{{R}_{s}} \qquad ( 8 )$$

where c₁, c₂, c₃, and c₄ are layout dependent. For square shape of inductor, c₁=1.27, c₂=2.07, c₃=0.18, and c₄=0.13. Davg is the average diameter, ρ is the fill ratio, n is number of turns, and μ₀ is the magnetic permeability of free space.

### External Readout Coil Design

The readout coil inductor is designed by soldering the coil with a SMA connector, which is connected to the Vector Network Analyzer (VNA) to measure the phase and amplitude of frequency response. The inductance of the external readout coil is given:

$$L = ( {d}^{2} * {n}^{2} ) / ( 18 d + 40 l ) \qquad ( 9 )$$

where L is inductance in micro-Henrys, d is the coil diameter in inches, 1 is the coil length in inches, and n is number of turns.

Using the above equation, the inductance of readout coil inductor with different number of turns can be obtained. FIG. 1E is a graph demonstrating inductance vs. the number of turns of readout coil inductors. As shown, the inductance increases as the number of turns increases. The performance of inductance versus number of turns is linear since the number of turns increases, the coil length increases.

### Example #1

The readout coil inductor is made of copper line. By using Vector Network Analyzer (VNA), the s1p files of each coil inductor can be obtained. After tuning the L, C and R. the Impedances are matched at 629.2 MHz, which is shown in FIG. 1F. The red curve in FIG. 1F shows the measurement result and the blue curve represents the simulation result that is built from BVD (Butterworth Van-Dyke) model of inductor to match the result of measurement. Using equations in the simulation result, the quality factor (Q), inductance, and resistance can be obtained. The self-resonant frequency is 634.9 MHz, and the value of Q is 156.69 at 120 MHz. The operating frequency is usually 5 to 10 times lower than self-resonant frequency since we can find a better Q value at that frequency range.

FIG. 1F are graphs showing measurement and simulation results of readout coil inductor with number of turns of 2 (The red curve is measurement result, and the blue curve is simulation result). After using the Advanced Design System (ADS) to do the simulation, the resonant frequencies for different number of turns can be obtained. The resistance increases with the number of turns increases, and the resistance depends on the length of coil. Meanwhile, the inductance increases with the number of turns increases. However, the capacitance keeps constant because it comes from the SMA connector. Then, the resonant frequency decreases with the number of turns increasing due to the increment of inductance.

With the number of turns increases, the calculation inductance and measurement inductance increase. The calculation inductance is lower than the measurement inductance since there is a small distance in between the different loops of inductor coil when the inductor is made. According to this equation, L=(d²*n²)/(18 d +40 l), with the coil length increases, the value of inductance decreases.

### Example Methods of Fabrication

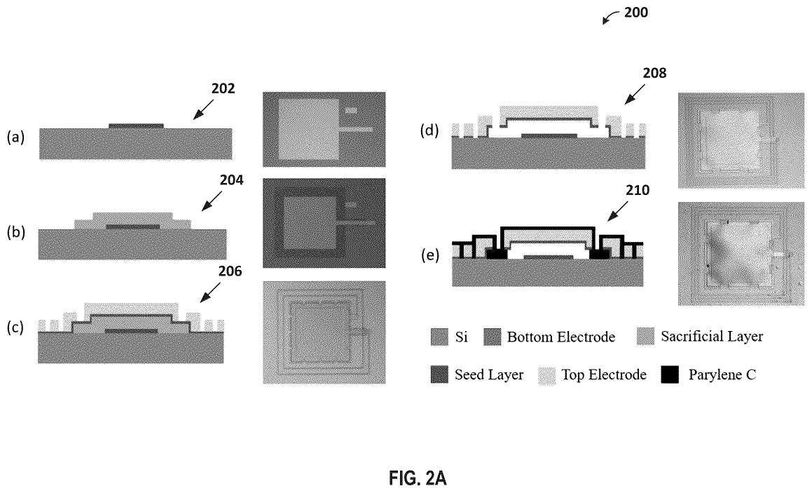

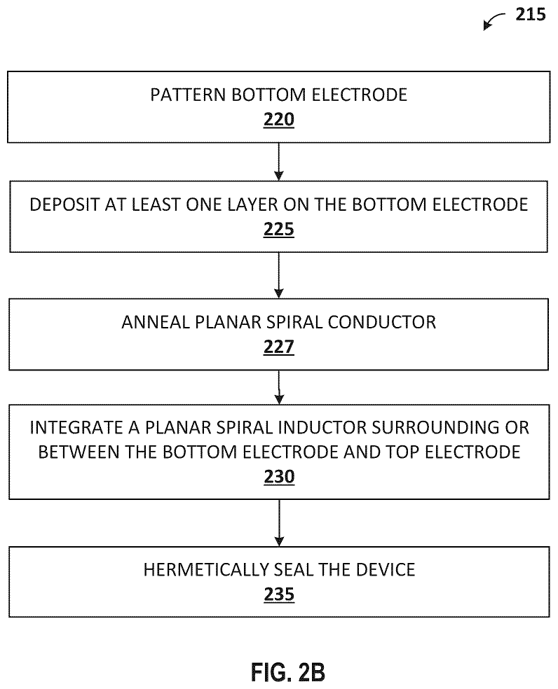

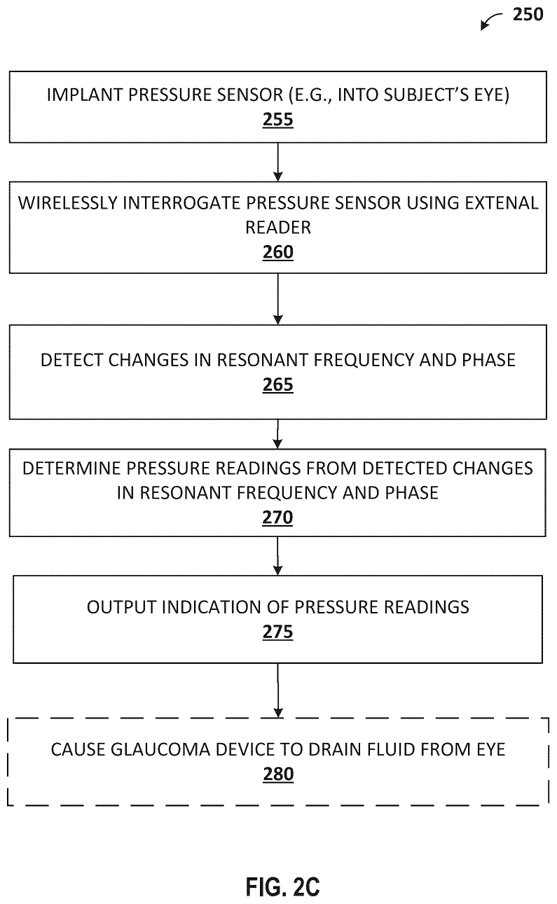

FIG. 2A and FIG. 2B show proposed fabrication processes 200, 215 for an exemplary IOP sensor/MEMS capacitive pressure sensor, in accordance with certain embodiments of the present disclosure.

The sensor can be a sealed or encapsulated parallel-plate capacitor with a deformable membrane that is configured to be implanted in a subject's body (e.g., eye, bladder, brain, or the like). As described herein, the pressure sensor can comprise an LC tank circuit including a spiral inductor and a variable capacitor or an inductor electrically coupled to the capacitor to form a resonant LC circuit. In some implementations, a soft-magnetic ferrite material is positioned (e.g., disposed, located) between the implanted pressure sensor and the external reader to enhance magnetic coupling. In some embodiments, the sensor is configured as or defines an array of sensors (e.g., 101a, 101b, 101c, 101d, 101e, illustrated in FIG. 1A). A parallel array of varying-gap membrane MEMS capacitors can be designed and implemented to a larger scale (e.g. a 10×10 array or even larger array size). In another example, one sensor can be implanted in each eye (e.g., a first sensor in the right eye and a second sensor in the left eye). Employing an array of sensors can serve to scale up the total capacitance change via parallel combinations of gap-varying capacitors into a larger array (e.g., the 10×10 array mentioned above). This disclosure contemplates the use of various materials to form the exemplary pressure sensor including nickel, copper, gold, silicon, polymers, and so on, as described in connection with FIG. 2A.

The example process 200 starts off at step 202 where a bottom electrode is patterned by sputtering and lift-off.

At step 204, a sacrificial layer (SiO2, photoresist, or other choice of sacrificial material) is deposited by Plasma Enhanced Chemical Vapor Deposition (PECVD) (or spin coating for the photoresist based sacrificial layer as well as other deposition techniques for other sacrificial layer material choices) and patterned by Deep Reactive Ion Etching (DRIE) or UV photolithography if a photoresist is chosen as the sacrificial layer. With other choices of sacrificial material, the process can vary as long as it can effectively define the sacrificial layer to the desired pattern.

At step 206, a seed layer is deposited by sputtering and the capacitor's top electrode and an integrated spiral inductor is patterned by nickel electroplating within patterned micro-molding.

At step 208 the seed layer is etched by Cu etchant and DRIE, followed by device release process by buffered oxide etch (BOE) or HF or photoresist stripper solution if a photoresist is used as the chosen sacrificial layer.

At step 210, the device is hermetically sealed, for example, by parylene C deposition.

Referring now to FIG. 2B, another example process 215 for fabricating a pressure sensor in accordance with certain embodiments of the present disclosure is provided.

At step 220, the method 215 includes patterning a bottom electrode.

At step 225, the method 215 includes depositing at least one layer on the bottom electrode. For example, the method 215 can include depositing a sacrificial layer and/or seed layer on the bottom electrode.

At step 227, the method 215 includes annealing a planar spiral conductor. Annealing the planar spiral inductor can comprise using rapid thermal processor (RTP) annealing or localized annealing through Joule heating via applied current. The quality factor of the microfabricated planar spiral inductor is low, which limit the resolution of the intraocular pressure sensors generally composed of inductor and variable capacitor. FIG. 1G is a graph demonstrating that the quality factor of the planar spiral inductor can be improved by annealing (rapid thermal processor (RTP) annealing or localized annealing through Joule heating via applied current).

At step 230, the method 215 includes integrating a planar spiral inductor surrounding or between the bottom electrode and a top electrode.

At step 235, the method 215 includes hermetically sealing the pressure sensor/device such that an air gap is present between the bottom electrode and the top electrode. In some implementations, the pressure sensor can be sealed using parlyene C deposition or other methods to form a biocompatible passivation layer.

### Example #2

In one implementation, the process (e.g., process 200, 215) can begin with patterning the bottom electrode of the MEMS membrane capacitor by sputtering and lift-off. A layer of oxide is deposited on top to act as the sacrificial layer. The second seed layer is deposited for nickel electroplating to form both spiral inductor and the top electrode of the MEMS membrane capacitor. For the nickel electroplating process, a solution of nickel sulfamate was used to achieve low stress and high ductility. The solution was mixed with boric acid to keep the PH value between 3.5 and 4.5 to reduce the roughness and pitting, while nickel chloride and sodium lauryl sulfate were added to improve the conductivity and to improve the brightness, respectively. The wafer is connected to the cathode or the positive electrode of the current source, while a pure Nickel plate is connected to the anode. In the electroplating process when the current source is running, the Ni2+ ions are attracted and deposited on the cathode. Once the electroplating is performed, the seed layer is etched by Cu etchant and DRIE, followed by device release process by buffered oxide etch (BOE) or HF. Finally, a conformal layer of parylene C is deposited on top of the device to hermetically seal the capacitive membrane transducer. The total number of masks for this proposed IOP sensor is only 4 because it is easier to fabricate the device with fewer masks. Meanwhile, the minimum alignment tolerance is 10 μm, thus resulting in lesser concern of misalignment. The fully released IOP sensor will be hermetically sealed by a layer of parylene C that provides an excellent barrier, while showing a very low permeability to moisture and gases.

### Example Method of Use

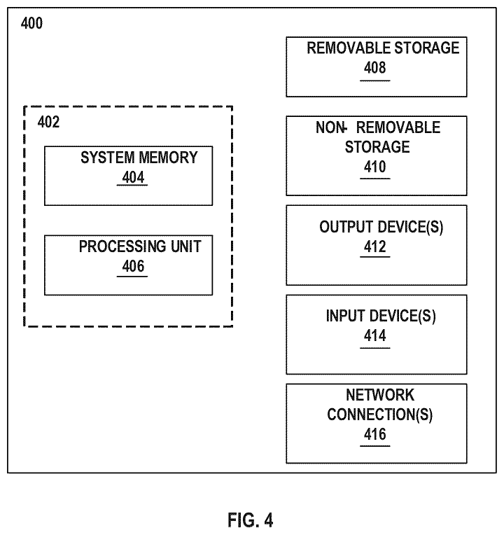

Referring now to FIG. 2C, a flowchart diagram depicting an example method 250 employing the pressure sensor described above, in accordance with various embodiments described herein is provided. This disclosure contemplates that the example method 250 can be at least partially performed using one or more computing devices (e.g., at least the configuration illustrated in FIG. 4 by box 402). In various implementations, the pressure sensor can be implanted elsewhere in a subject's body and may be configured for bladder, intracranial, or cerebrospinal fluid (CSF) pressure monitoring.

At step/operation 255, the method 250 includes implanting a pressure sensor, for example, into a subject's eye. The pressure sensor can be implanted in a subject's eye via at least one of: iris suturing, vitreous cavity anchoring, or a lens bag placement. As noted above, the pressure sensor can also be implanted in a subject bladder or brain. The pressure sensor can also be embedded in and/or embodied as part of a glaucoma drainage device or intraocular lens. An intraocular lens is capable of continuous intraocular pressure measurements upon implantation into the eye and a glaucoma drainage device is capable of continuous intraocular bleeding pressure measurements upon implantation into the eye.

At step/operation 260, the method 250 includes wirelessly interrogating the implanted pressure sensor using an external reader. For example, the pressure sensor can comprise an LC tank circuit or resonant LC circuit, and the external reader can be configured to wirelessly interrogate the LC tank circuit or resonant LC circuit via inductive coupling. In various implementations, the external reader detects changes in resonant frequency and phase corresponding with an induced deformation of the membrane resulting in capacitance changes corresponding to intraocular pressure variations. In some implementations, the operational distance between the pressure sensor and the external reader can be between 0 cm and 1 foot, and an example detection range can fall 0 mmHg and 50 mmHg, with a system resolution of at least 0.1 mmHg.

The external reader can comprise a signal processing circuit (e.g., closed loop oscillator) configured to convert the detected changes in resonant frequency and phase to pressure readings, and/or output or transmit an indication of the pressure readings at step/operation 275 below. As described above, at least one repeater coil or circuit can be positioned between the pressure sensor and external reader to improve signal strength therebetween.

At step/operation 265, the method 250 includes detecting changes in resonant frequency and phase. For example, capacitance changes corresponding to intraocular pressure variations can lead to (e.g., induce, elicit) a change in frequency and phase of the pressure sensor's LC tank circuit or resonant LC circuit.

At step/operation 270, the method 250 includes determining pressure readings from the detected changes in resonant frequency and phase while taking into account the sensor calibration.

At step/operation 275, the method 250 includes outputting an indication the pressure readings to a computing device and/or graphical user interface.

Optionally, at step/operation 280, the method 250 includes causing a glaucoma device operatively coupled to the pressure sensor to drain fluid from the eye (e.g., in response to detecting pressure readings that meet or exceed a predetermined value or range).

### Experimental Results

The research proposed herein aims to investigate the surgical insertion of the MEMS IOP sensor microchip coated with Parylene for encapsulation, to ensure biocompatibility, inside a PMMA based container as its hermetic package. This packaged IOP sensor is designed to be implanted in the anterior chamber of the eye or other regions of a subject's eye with the drainage device (can use silicone frame or drilling holes on the silicon substrate for suturing as well). Meanwhile, the external readout coil and vector network (impedance) analyzer used in our initial study can be replaced by a handheld close-loop oscillator-based mixed signal processor that can display the IOP eye pressure in mmHg unit to make it convenient for the target end users (patient at home) to enable real-time IOP monitoring. A readout circuit for accurate reading of the intraocular pressure induced variation of MEMS capacitive membrane sensor can eliminate the need of a bulky and costly vector network or impedance analyzer for the point-of-care measurement at home that has never been explored and demonstrated in prior works to our best knowledge. Without critical point-of-care, portable apparatus, it is impossible for real patients at home to use technology as they need the help of engineers to operate specialized test tools (e.g., vector network analyzer/impedance analyzer).

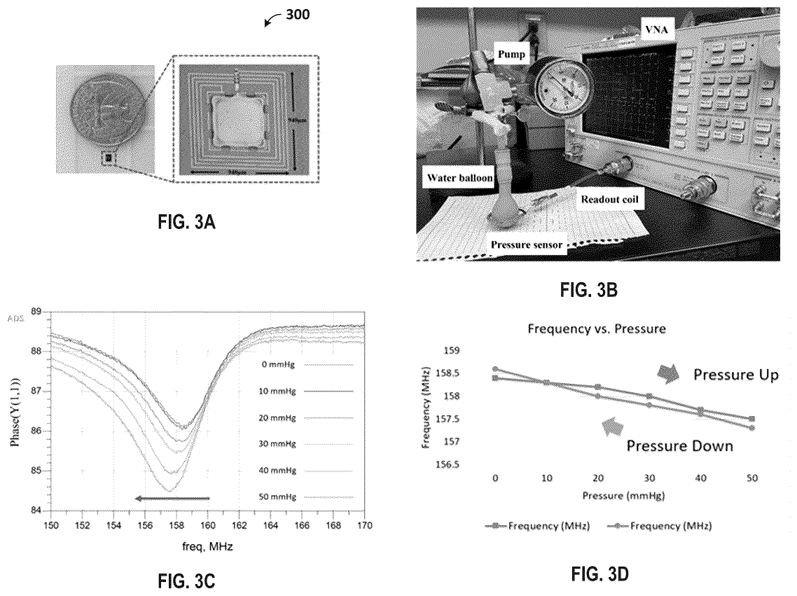

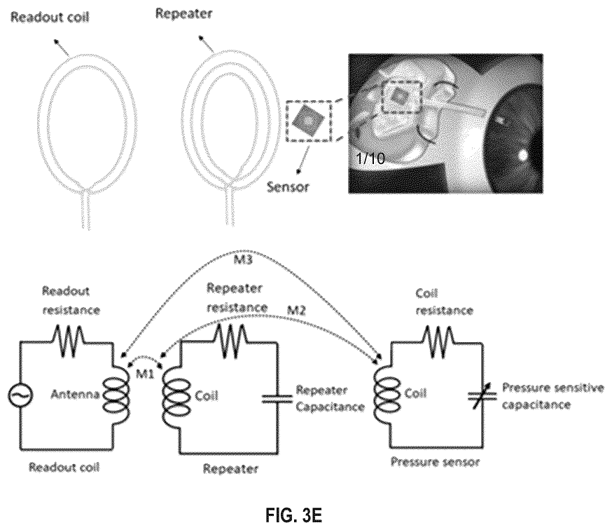

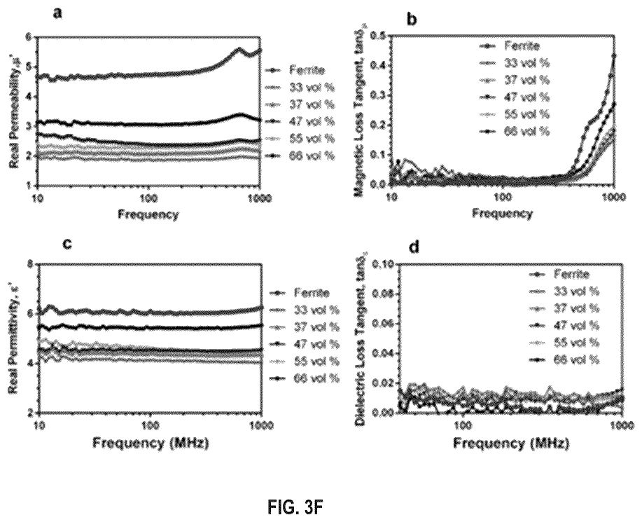

FIG. 3A is a zoom-in image of a fabricated IOP capacitive membrane sensor with a surrounding spiral inductor showing a size comparison of a sensor chip vs. a US quarter.

FIG. 3B depicts an initial sensor test scheme that has a water balloon and pump, a reference pressure sensor (for calibration), a sensor readout coil, a vector network/impedance analyzer, which tests the frequency response (frequency or phase drift) of the sensor under varied liquid pressure applied via a pumped water balloon as a media to simulate the IOP pressure. Due to the mutual coupling of the built-in spiral inductor and external readout coil, the vector network analyzer connected to the readout coil can accurately detect the change of the resonance frequency and phase from the implantable IOP sensor in a noncontact mode. The resonance frequency change and phase dip variation induced by varied liquid pressure is continuously detected by telemetry. Through inductive coupling, the external coil energizes the implantable IOP sensor that provides an impedance loading to the external coil that is accurately detected by a vector network analyzer. By monitoring the impedance change via the external coil due to mutual coupling with IOP sensor, it is possible to detect the sensor's frequency and phase change in real time.

FIG. 3C shows a measured frequency response of the IOP sensor via an external readout coil and a vector network analyzer showing an induced frequency and phase change, which are both linearly proportional to the liquid pressure applied through water balloon. Specifically, FIG. 3C shows a tested phase dip variation of the IOP sensor vs. liquid pressure of 0-50 mmHg. As seen by Eq. (3), the phase dip is maximized when the series resistance of the integrated inductor Rs is minimized, whereas a greater mutual inductance can be obtained if the IOP sensor's inductance Ls is increased. But, when Ls is increased by increasing the number of turns of the inductor, the parasitic capacitance increases as well, thus decreasing the self-resonance frequency to limit its frequency range. In general, the self-resonance frequency should be much higher (>3× higher) than the operation frequency. Thus, it is critical to lower the series resistance (Rs) of the sensor inductor for enhancing the detection limit (resolution) of the proposed sensor. Low Rs (high Q) integrated spiral inductors can be realized by depositing thicker and more conductive layers via optimized electroplating with low resistivity. For the proposed research, there are novel ideas related to readout circuitry (wireless telemetry) design that can be pursued to enhance IOP sensor's overall performance (resolution, repeatability independent of position/alignment of the external readout coil, and case of sensor calibration, etc.).

FIG. 3D shows an extracted resonance frequency variation in response to liquid pressure change between 0 and 50 mmHg. As the pressure increases, the resonant frequency decreases due to the increase of the membrane capacitance.

Following the pioneering prior work by researchers at MIT in 2007 for mid-range wireless power transfer [16,17] via a “source-relay(s)-device” topology, Zhang, et al. studied a similar method to extend long-range distance of inductive-capacitive (LC) passive wireless sensors via strongly coupled magnetic resonance (SCMR). In this paper, the use of repeater as intermediate relays to enhance the magnetic resonance will be exploited to improve the readout circuit. A coefficient matrix will be employed to analyze the effect of the repeater(s), which theoretically impacts frequency division and sensitivity attenuation. The novel wireless senor interrogation with repeaters can avoid sensitivity decay by adding two extra peak frequencies. In these prior works, variable capacitor (varactor)-based LC passive sensor uses up to 3 extra repeaters (LC resonators) for wireless telemetry measurements have been explored. The results indicate the long-range distance of passive wireless sensors with repeaters were improved by 180% without a penalty, such as sensitivity degradation observed in designs without the use of repeater(s) [18]. The key factor that limits readout distance is poor magnetic coupling between external readout coil and integrated inductor alongside the capacitive sensor. By strategically adding intermediate LC resonators (repeaters) that receive the magnetic field from the RF source (readout coil) and then relay the magnetic field to the receiver (IOP sensor), strongly couple magnetic resonance can be realized, therefore enabling longer wireless interrogation distances or better sensor repeatability.

FIG. 3E is a schematic diagram of a LC passive wireless sensor with a repeater strategically applied to proposed IOP sensor for relaxing the sensitivity of readout coil to IOP sensor alignment while fortifying output signal. In FIG. 3E, the repeater (LC resonator) is strategically designed and positioned in between the external interrogation coil and the integrated inductor of the sensor.

The effectiveness of the repeaters in wireless interrogated sensors has been demonstrated recently [19]. However, the added repeaters also create multiple peak frequencies and could cause sensitivity degradation. Thus, the coefficient matrix method is used to derive two split peak frequencies in the LC passive sensor system with an added repeater. Interrogation methods to avoid sensitivity attenuation will be adopted by considering the two peak frequencies. The equivalent circuit of a LC passive wireless sensor system with one repeater is shown in FIG. 3E, M1, M2 and M3 are mutual inductance between readout coil and repeater, sensor and repeater, and readout coil and sensor, respectively. A varactor-based LC passive sensor can be used to verify and maximize the effect of the repeater. Similarly, varactor-based passive sensors have been reported to wirelessly measure pH [20] or biopotential [21]. To our best knowledge, the proposed work is the first attempt to incorporate repeater(s) into an IOP sensor wireless telemetry. In prior work, a layer called “B-field sheet” is added in between transmitter and receiver inductors to strengthen the magnetic flux density. FIG. 3E shows simulated magnetic field distribution between transmit and receive coils while showing the effect of a repeater in between them. To enhance the magnetic coupling for much longer distances, added repeater(s) that gets transmitted magnetic field by the transmitter coil and then transfers the field to the receiver coil as shown in FIG. 3E. It is observed the magnetic field strength has been greatly enhanced with added repeater at an optimized position.

To improve the overall performance of wireless pressure sensor telemetry, a newly developed soft magnetic (NiCuZn) ferrite material will be used in the proposed work. NiCuZn ferrite samples show enhancement in magnetic permeability, while exhibiting low magnetic loss up to 1 GHz. In our recent prior work, NiCuZn soft magnetic ferrite sheets have been fabricated by using in-house synthesized NiCuZn powders for wireless power transfer (WPT) applications [22]. A study was conducted to develop a standard operation procedure for preparation of the soft magnetic NiCuZn ferrite material with well-characterized magnetic and loss properties [22].

Nowadays, Near-Field Communication (NFC) technology is widely used in a variety of wireless communications [23,24]. Wireless power transfer techniques use inductive coupling to transfer power wirelessly through the magnetic induction generated by paired coils [25,26]. The frequency dispersion of complex permeability impacts the efficiency of wireless power transfer [27]. For the proposed IOP sensor wireless interrogation, the insertion of a thin sheet made of soft magnetic ferrites between the paired transmitter and receiver coils lowers the eddy currents and extends the range of the magnetic fields, thus enhancing the sensor readout efficiency. To enhance the overall performance of wireless telemetry, a thin NiCuZn ferrite sheet of high permeability and low magnetic loss is desired [28]. In-house developed Ni—Cu—Zn ferrites have exhibited promising soft magnetic properties for high frequency applications due to its high permeability, low loss and high resistivity at RF and microwave frequencies up to 1 GHz [29-31].

FIG. 3F are graphs showing tested magnetic permeability and loss tangent of NiCoZn soft magnetic ferrite sheet that can be used along with repeaters to enhance sensor readout via strongly coupled magnetic resonance. In particular, FIG. 3F presents tested magnetic and loss properties of in-house synthesized NiCuZn ferrites, which exhibits magnetic permeability of 2.4-2.6, permittivity of 4.4, and magnetic and dielectric loss tangents of 0.03 and 0.006 at frequencies up to 1 GHz, respectively. The unprecedented soft magnetic properties are deemed to be instrumental for further enhancing the readout signal strength of the proposed sensor wireless telemetry through strongly coupled magnetic resonance by using added repeater coil(s).

### Computing Devices and Methods of Use

This disclosure contemplates that the analog signal gathered by the readout circuit described herein can be further processed by a portable gadget or device with a basic level of digital signal processing capabilities, which can be used as a plug-in ready unit to a much more popular and sophisticated microcomputer, smart phone or a laptop computer with some software (firmware) developed to display the tested IOP pressure reading to the end users (patients at home).

It should be appreciated that the logical operations described herein with respect to the various figures may be implemented (1) as a sequence of computer-implemented acts or program modules (i.e., software) running on a computing device (e.g., the computing device described in FIG. 4), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special-purpose digital logic, and any combination thereof. It should also be appreciated that more or fewer operations may be performed than shown in the figures and described herein. These operations may also be performed in a different order than those described herein.

Referring to FIG. 4, an example computing device 400 upon which embodiments of the present disclosure may be implemented is illustrated. It should be understood that the example computing device 400 is only one example of a suitable computing environment upon which embodiments of the present disclosure may be implemented. Optionally, the computing device 400 can be a well-known computing system including, but not limited to, personal computers, servers, handheld or laptop devices, multiprocessor systems, microprocessor-based systems, personal network computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, the computing device 400 typically includes at least one processing unit 406 and system memory 404. Depending on the exact configuration and type of computing device, system memory 404 may be volatile (such as random-access memory (RAM)), non-volatile (such as read-only memory (ROM), flash memory, etc.), or some combination of the two. This most basic configuration is illustrated in FIG. 4 by the dashed line 402. The processing unit 406 may be a standard programmable processor that performs arithmetic and logic operations necessary for the operation of the computing device 400. The computing device 400 may also include a bus or other communication mechanism for communicating information among various components of the computing device 400.

Computing device 400 may have additional features/functionality. For example, the computing device 400 may include additional storage such as removable storage 408 and non-removable storage 410 including, but not limited to magnetic or optical disks or tapes. Computing device 400 may also contain network connection(s) 416 that allow the device to communicate with other devices. Computing device 400 may also have input device(s) 414 such as a keyboard, mouse, touch screen, etc. Output device(s) 412, such as a display, speakers, printer, etc., may also be included. The additional devices may be connected to the bus in order to facilitate communication of data among the components of the computing device 400. All these devices are well-known in the art and need not be discussed at length here.

The processing unit 406 may be configured to execute program code encoded in tangible, computer-readable media. Tangible, computer-readable media refers to any media that is capable of providing data that causes the computing device 400 (i.e., a machine) to operate in a particular fashion. Various computer-readable media may be utilized to provide instructions to the processing unit 406 for execution. Example of tangible, computer-readable media may include but is not limited to, volatile media, non-volatile media, removable media and non-removable media implemented in any method or technology for storage of information such as computer-readable instructions, data structures, program modules or other data. System memory 404, removable storage 408, and non-removable storage 410 are all examples of tangible computer storage media. Examples of tangible, computer-readable recording media include but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 406 may execute program code stored in the system memory 404. For example, the bus may carry data to the system memory 404, from which the processing unit 406 receives and executes instructions. The data received by the system memory 404 may optionally be stored on the removable storage 408 or the non-removable storage 410 before or after execution by the processing unit 406.

It should be understood that the various techniques described herein may be implemented in connection with hardware or software or, where appropriate, with a combination thereof. Thus, the methods and apparatuses of the presently disclosed subject matter, or certain embodiments or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, for example, through the use of an application programming interface (API), reusable controls, or the like. Such programs may be implemented in a high-level procedural or object-oriented programming language to communicate with a computer system. However, the program(s) can be implemented in assembly or machine language if desired. In any case, the language may be a compiled or interpreted language, and it may be combined with hardware implementations.

In one embodiment, disclosed herein is a non-transitory computer-readable storage medium comprising instructions that, when executed, cause at least one processor to perform the method of any preceding embodiments.

Although certain implementations may refer to utilizing aspects of the presently disclosed subject matter in the context of one or more stand-alone computer systems, the subject matter is not so limited but rather may be implemented in connection with any computing environment. For example, the components described herein can be hardware and/or software components in a single or distributed systems, or in a virtual equivalent, such as, a cloud computing environment. Still further, aspects of the presently disclosed subject matter may be implemented in or across a plurality of processing chips or devices, and storage may similarly be affected across a plurality of devices. Such devices might include personal computers, network servers, and handheld devices, for example.

The following patents, applications and publications as listed below and throughout this document are hereby incorporated by reference in their entirety herein.

### REFERENCE LIST

[1] Y. H. Kwon, J. H. Fingert, M. H. Kuehn, and W. L. M. Alward, “Primary open-angle glaucoma,” N. Engl. J. Med., vol. 360, no. 11, pp. 1113-1124 March 2009.
[2] S. Resnikoff, D. Pascolini, D. Etya'ale, et al, “Global data on visual impairment in the year 2002,” Bulletin of the World Health Organization, vol. 82, no. 11, pp.844-851, 2004.
[3] A. S. Frank and P. A. Netland, “The evolution of glaucoma drainage implants,” Journal of ophthalmic & vision research, vol. 13, no. 4, pp. 498-500, 2018.
[4] K. S. Arora, A. L. Robin, K. J. Corcoran, S. L. Corcoran, and P. Y. Ramulu, “Use of various glaucoma surgeries and procedures in medicare beneficiaries from 1994 to 2012,” Ophthalmology, vol. 122, no. 8, pp. 1615-1624 August 2015.
[5] H. A. Quigley and A. T. Broman, “The number of people with glaucoma worldwide in 2010 and 2020,” Br. J. Ophthalmol., vol. 90, no. 3, pp. 262-267, March 2006.
[6] J. C. Tsai, “High eye pressure and glaucoma,” Glaucoma Research Foundation, September 2015.
[7] C. C. Collins, “Miniature passive pressure transensor for implanting in the eye,” IEEE Trans. Biomed. Eng., vol. BME-14, no. 2, pp. 74-83, April 1967.
[8] P.-J. Chen, D. C. Rodger, S. Saati, M. S. Humayun, and Y.-C. Tai, “Microfabricated implantable parylene-based wireless passive intraocular pressure sensors,” Journal of Micromechanics and Microengineering, vol. 17, pp. 1931-1938 August 2007.
[9] P.-J. Chen, S. Saati, R. Varma, M. S. Humayun, and Y.-C. Tai, “Wireless intraocular pressure sensing using microfabricated minimally invasive flexible-coiled LC sensor implant,” Journal of Microelectromechanical Systems, vol. 19, no. 4, pp. 721-734, August 2010.
[10] B. Kang, H. Hwang, S. H. Lee, “A wireless intraocular pressure sensor with variable inductance using a ferrite material,” Journal of semiconductor technology and science, vol. 13, no. 4, August 2013.
[11] P. Zeng, Q. Cui, M. Wu, P. Chen and M. M. Cheng, “Wireless and continuous intraocular pressure sensors using transparent graphene,” 2016 IEEE Sensors, pp. 1-3, November 2016.
[12] J. O. Akar, T. Akin, and K. Najafi, “A wireless batch sealed absolute capacitive pressure sensor,” Sensors and Actuators A: Physical, vol 95, pp. 29-38, January 2001.
[13] S. A. Shabalovskaya, “On the nature of the biocompatibility and on medical applications of NiTi shape memory and superelastic alloys,” Bio-Medical Materials and Engineering, vol. 6, no. 4, pp. 267-289, 1996.
[14] K. B. Balavalad, B. Mudhol, B. G. Sheeparamatti, and P.B. Balavalad, “Comparative analysis on design and simulation of perforated MEMS capacitive pressure sensor,” International Journal of Engineering Research & Technology (IJERT), vol. 4, no. 7, July 2015.
[15] P. Pirouznia and B. A. Ganji, “Analytical optimization of high performance and high quality factor MEMS spiral inductor,” Progress In Electromagnetics Research M, vol. 34, pp. 171-179, 2014.
[16] KURS, André et al. “Wireless Power Transfer via Strongly Coupled Magnetic Resonances.” Science (American Association for the Advancement of Science) 317.5834 (2007): 83-86.
[17] Ahn, Dukju, and Songcheol Hong. “A Study on Magnetic Field Repeater in Wireless Power Transfer.” IEEE transactions on industrial electronics (1982) 60.1 (2013): 360-371.
[18] Zhang, Cong, Li-Feng Wang, and Qing-An Huang. “Extending the Remote Distance of LC Passive Wireless Sensors via Strongly Coupled Magnetic Resonances.” Journal of micromechanics and microengineering 24.12 (2014): 125021.
[19] Sanz, Diego A. et al. “Passive Resonators for Wireless Passive Sensor Readout Enhancement.” Applied physics letters 103.13 (2013): 133502.
[20] Bhadra, Sharmistha et al. “A Wireless Passive Sensor for Temperature Compensated Remote pH Monitoring.” IEEE sensors journal 13.6 (2013): 2428-2436.
[21] Riistama, J. et al. “Totally Passive Wireless Biopotential Measurement Sensor by Utilizing Inductively Coupled Resonance Circuits.” Sensors and actuators. A. Physical. 157.2 (2010): 313-321.
[22] Lathiya, Poonam, Marcel Kreuzer, and Jing Wang. “RF Complex Permeability Spectra of Ni—Cu—Zn Ferrites Prepared Under Different Applied Hydraulic Pressures and Durations for Wireless Power Transfer (WPT) Applications.” Journal of magnetism and magnetic materials 499 (2020): 166273—. Web.
[23] S. S. Lau, “Practical design of 13.56 MHz Near Field Communication (NFC) and Radio Frequency Identification (RFID) antenna using ferrite sheet on metallic surface by Network Analyzer,” 2015 IEEE International Conference on Industrial Engineering and Engineering Management (IEEM), 2015, pp. 1362-1366, doi: 10.1109/IEEM.2015.7385870.
[24] Wu, Xiaohan et al. “Influence of Particle Size on the Magnetic Spectrum of NiCuZn Ferrites for Electromagnetic Shielding Applications.” Journal of magnetism and magnetic materials 401 (2016): 1093-1096. Web.
[25] M. Gebhart, R. Neubauer, M. Stark and D. Warnez, “Design of 13.56 MHz Smartcard Stickers with Ferrite for Payment and Authentication,” 2011 Third International Workshop on Near Field Communication, 2011, pp. 59-64, doi: 10.1109/NFC.2011.14.
[26] Yan, Shuoqing et al. “Preparation and Characterization of Ferrite with Co Substituted NiCuZn Sheets Application for 13.56 MHz Radio Frequency Identification Communication.” Journal of applied physics 115.17 (2014): 17—. Web.
[27] W. Lee, Y.-K. Hong, J. Park, J. Lee, I.-S. Baek, N.-P. Hur, W.-M. Seong, S.-O. Park. “A simple wireless power charging antenna system: evaluation of ferrite sheet,” IEEE Trans. Magn. 53 (2017) 1-5.
[28] C. Stergiou, E. Eleftheriou, V. Zaspalis, “Enhancement of the near-field UHF RFID with ferrite substrates,” IEEE Trans. Magn. 48 (2012) 1497-1500.
[29] H. Su, H. Zhang, X. Tang, Z. Zhong, Y. Jing, “Analysis of low-temperature-fired NiCuZn ferrites for power applications,” Mater. Sci. Eng. B 162 (2009) 22-25.
[30] T. Tsutaoka, “Frequency dispersion of complex permeability in Mn—Zn and Ni—Zn spinel ferrites and their composite materials,” J. Appl. Phys. 93 (2003) 2789-2796.
[31] Z. Zheng, Q. Feng, Q. Xiang, Z. Di, V.G. Harris, “Low-loss NiZnCo ferrite processed at low sintering temperature with matching permeability and permittivity for miniaturization of VHF-UHF antennas,” J. Appl. Phys. 121 (2017) 063901.

1. A wireless intraocular pressure sensing system comprising:
a pressure sensor including a sealed or encapsulated parallel-plate capacitor with a deformable membrane, the pressure sensor being configured to be implanted in a subject's eye;
an external reader outside the subject's eye configured to wirelessly interrogate the pressure sensor and detect changes in resonant frequency and phase corresponding with an induced deformation of the membrane resulting in capacitance changes corresponding to intraocular pressure variations;
at least one repeater coil or circuit positioned between the implanted pressure sensor and the external reader configured to improve signal strength between the pressure sensor and the external reader, wherein the external reader comprises a readout circuit and a signal processing circuit configured to:
convert the detected changes in resonant frequency and phase to pressure readings, and
output or transmit an indication of the pressure readings.

2. The system of claim 1, wherein the pressure sensor comprises an LC tank circuit including a spiral inductor and a variable capacitor connected in series or parallel configuration, or an inductor electrically coupled to the capacitor to form a resonant LC circuit.

3. The system of claim 2, wherein the external reader is configured to wirelessly interrogate the LC tank circuit or resonant LC circuit via inductive coupling.

4. The system of claim 2, wherein the capacitance changes lead to a change in frequency and phase of the LC tank circuit or resonant LC circuit.

5. The system of claim 1, wherein the signal processing circuit is configured to output or transmit the indication of the pressure readings to a computing device or graphical user interface.

6. The system of claim 5, wherein the computing device is configured to transmit a control indication to cause an implanted glaucoma drainage device to drain fluid from the subject's eye.

7. The system of claim 1, wherein the signal processing circuit and readout circuit comprise a closed-loop oscillator to form a point-of-care portable test module.

8. The system of claim 1, further comprising:
a soft-magnetic ferrite material positioned between the implanted pressure sensor and the external reader to enhance magnetic coupling.

9. The system of claim 1, wherein the pressure sensor comprises a Micro-Electro-Mechanical Systems (MEMS) capacitive pressure sensor.

10. The system of claim 9, wherein the MEMS capacitive pressure sensor comprises an array of pressure sensors to form parallel array of varying-gap capacitors.

11. The system of claim 1, wherein the pressure sensor comprises a biocompatible passivation layer.

12. The system of claim 1, wherein the pressure sensor comprises nickel, gold, silicon, or polymer.

13. The system of claim 1, wherein the pressure sensor is embedded in or embodied as a glaucoma drainage device or intraocular lens.

14. The system of claim 1, wherein an operational distance between the pressure sensor and the external reader is between 0 cm and 1 foot.

15. The system of claim 1, wherein the system detection range is between 0 mmHg and 50 mmHg, and wherein the system resolution is at least 0.1 mmHg.

16. The system of claim 1, wherein the pressure sensor is configured for implantation via at least one of: iris suturing, vitreous cavity anchoring, or a lens bag placement.

17. A method of manufacturing a pressure sensor, the method comprising:
patterning a bottom electrode;
depositing a plurality of layers on the bottom electrode;
annealing a planar spiral inductor;
integrating the planar spiral inductor surrounding or between the bottom electrode and a top electrode; and
hermetically sealing the pressure sensor, wherein an air gap is present between the bottom electrode and the top electrode.

18. The method of claim 17, wherein the pressure sensor is hermetically sealed using parylene C deposition or other bio-compatible thin film deposition.

19. The method of claim 17, wherein annealing the planar spiral inductor comprises using rapid thermal processor (RTP) annealing or localized annealing through Joule heating via applied current.

20. A method of monitoring intraocular pressure comprising:
implanting a pressure sensor into a subject's eye;
wirelessly interrogating the pressure sensor using an external reader;
detecting changes in resonant frequency and phase corresponding to intraocular pressure variations;
determining pressure readings from the detected changes in resonant frequency and phase; and
outputting or transmitting an indication of the pressure readings.

21-37. (canceled)
